(19) United States
(12) Patent Application Publication
(10) Pub. No.: US 2021/0401609 A1
(43) Pub. Date: Dec. 30, 2021

(54) Adjustable Brace

(71) Applicant: Joshua Reyna, San Antonio, TX (US)

(72) Inventor: Joshua Reyna, San Antonio, TX (US)

(21) Appl. No.: 17/362,546

(22) Filed: Jun. 29, 2021

Related U.S. Application Data
(60) Provisional application No. 63/045,987, filed on Jun. 30, 2020.

(51) Int. Cl. A61F 5/02; A61F 5/32

(52) U.S. Cl. CPC A61F 5/024; A61F 5/32

(57) ABSTRACT

An adjustable brace with a grid of adjustment pins threadedly enclosed within threaded pin apertures. The adjust pins have a domed cushion on the interior side of the brace. The adjustment pins are adjusted towards and away from the user's back as desired by rotating the pin shafts. The pin shafts are adjusted to customize the amount and location of pressure the brace applies on the user.

### CROSS-REFERENCES TO RELATED APPLICATIONS

This application claims the benefit of provisional patent application No. 63/045,987.

### STATEMENT REGARDING FEDERALLY SPONSORED RESEARCH OR DEVELOPMENT

Not applicable.

### BACKGROUND OF THE INVENTION

### 1. Field of the Invention

Braces are commonly used to provide support or pain management to different parts of the body. Braces exist for, inter alia, necks, backs, knees, elbows, calves, and ankles. Braces typically comprise an attachment mechanism such as straps and a support element that applies pressure on the user's body.

Back braces are commonly used to provide back support for users. Back braces can be used prophylactically to avoid injuries and/or as pain management. Cloth or nylon back braces that wrap around the user's lower back are common. The wrap style braces generally have a cushion or ridged support section located on the users' back and an attachment mechanism in the front for securing the brace.

### SUMMARY OF THE INVENTION

The adjustable brace allows the user to target pressure to desired locations on the user's body. The adjustable brace has a series of pressure pins that the user can tighten and loosen to customize the location and amount of pressure.

### BRIEF SUMMARY OF THE DRAWINGS

FIG. 1 is an exterior view of a first embodiment of an adjustable back brace.

FIG. 2 is an interior view of the first embodiment of an adjustable back brace.

FIG. 3 is a perspective view of a first embodiment of an adjustment pin.

FIG. 4 is a sectional view of the first embodiment of an adjustment pin inserted through a grid channel.

FIG. 5 is a perspective view of the first embodiment of an adjustable back brace in use.

### DETAILED DESCRIPTION

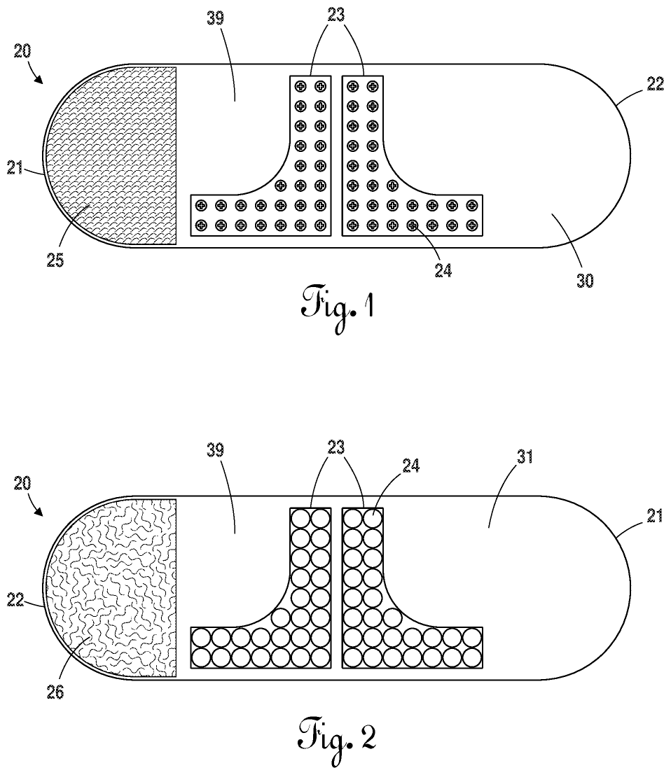

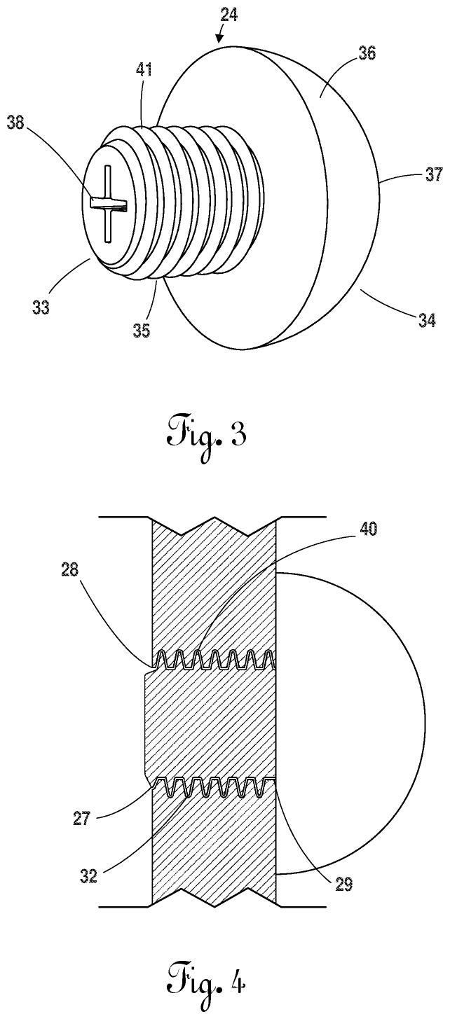

Referring to FIGS. 1 and 2, the adjustable back brace 20 comprises a band 39, pin grid 23, and adjustment pins 24.

The band 39 has a first end 21 and a second end 22. The band has an exterior side 30 and an interior side 31. The exterior side 30 of the band 39 has a first Velcro® section 25 proximate the first end 21 of the band 39. The interior side 31 of the band 39 has a second Velcro® section 26 proximate the second end 22 of the and 39. The band 39 can be cloth, nylon, or other suitable material. In other embodiments, other suitable attachment means than Velcro® could be used.

A pin grid 23 is integral the center of the band 39 between the exterior side 30 of the band 39 and the interior side 31 of the band 39. In one embodiment, the pin grid 23 is L-shaped. In other embodiments, the pin grid 23 can be tapered, square, or other suitable shapes.

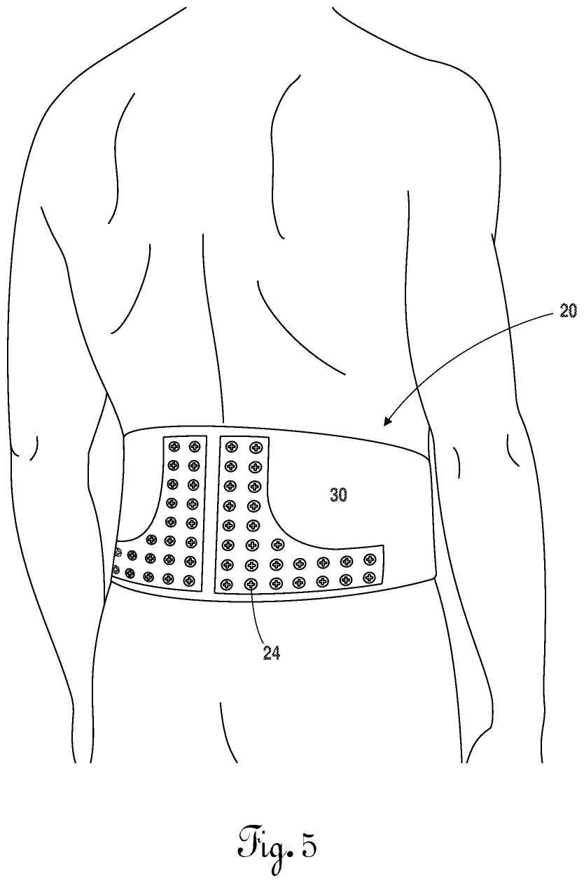

Referring to FIGS. 1, 2, and 4, the pin grid 23 comprises a plurality of pin apertures 27 that extend through the pin grid 39 between the exterior side 30 and interior side 31 of the band 39. The pin apertures 27 have an exterior opening 28 and an interior opening 29. A pin aperture channel 32 is formed between the exterior opening 28 and interior opening 29 of the pin apertures 27. The interior of the pin channel 32 is threaded 40. Those skilled in the art understand the number and location of pin apertures 27 can be customized as desired.

Referring to FIG. 3, The adjustment pins 24 have an exterior end 33, an interior end 34, a shaft 35, and a head 36. The exterior end 33 of the adjustment pins is generally circular with slots 38 for a screwdriver. The shaft 25 has threads 41. In one embodiment, the head 36 has a significantly larger diameter than the shaft 25 and comprises a dome shaped cushion 37 on the end. Those skilled in the art understand that the head 36 of the pin 24 can be customized to meet the user's desires. For example, the pin head 36 could be hard plastic, rubber, or a different shape. In some embodiments, the head 36 may be the same diameter as the rest of the pin 24.

The exterior diameter of the pin shafts 35 is barely smaller than the interior diameter of the pin aperture channels 32. The diameter of the pin head 36 is larger than the diameter of the interior and exterior aperture openings 28, 29.

The pin shafts 35 are enclosed within the pin apertures 27 and secured thereto by a thread fit between the threads 40 of the aperture channel 32 and the threads 41 of the pin shaft 35. The interior end 34 of the pin 24, head 36, and dome shaped cushion 37 are located on the interior side of the adjustable back brace 20.

Those skilled in the art understand the number and location of pins 24 and pin apertures 27 can be customized. Those skilled in the art further understand that not every pin aperture 27 needs a pin 24. The user may desire pins in only a select number or location of available apertures 27.

The function of the adjustable back brace is described with reference to FIGS. 1-5. The brace 20 is positioned so that the grid 23 is aligned with the part of the body to which the user wishes to apply pressure. In FIG. 5, the brace 20 is shown being worn by a user to apply pressure to the user's lower back.

The location and amount of pressure the brace 20 applies to the user is customized by adjusting the pins 24 to apply different amounts of pressure at different locations. In one embodiment, a standard screwdriver is used to rotate the pins 24 within the pin apertures 27. Those skilled in the art understand that other suitable tightening devices could be used, such as an Allen wrench or a hex key.

The threading 41 from the pin shaft 35 and threading 40 from the aperture channel 32 cause the pin 24 to incrementally move forward and backward within the channel 32 when the pin 24 is rotated. When a pin 24 is rotated inward toward the user, that pin 24 pushes the dome shaped cushion 37 on the pin head 36 toward and into the user's back. When a pin 24 is rotated inward toward the user, that pin 24 applies pressure on the user's back.

The user can rotate the screwdriver in the reverse direction to incrementally move the pin 24 towards the exterior of the brace 20, thereby lessening the pressure that pion 24 applies on the user.

In other words, by turning a screw clockwise, the user is pushing a ball like pin into his or her muscle to apply a controlled force in order to decompress the muscle. By rotating it counterclockwise the user is withdrawing the amount of control force that is being applied to the muscle. The user can pick and choose which pins 24 need to be oriented toward the user, therefore applying the most pressure, and which pins 24 need to be oriented closer to the band 39 therefore applying less pressure.

The user can also remove pins 24 from the brace 20 altogether, creating areas where no pressure is applied.

The brace 20 is designed to have pins 24 in the area(s) where your back muscles are located. Having multiple ball like pins that cover the back muscle you will able to apply multiple points of pressure to your back or lower back at one time. The ball like pins will be Extracted and retracted out of the back brace, allowing the ball like pins to apply pressure to the affected muscle area.

The adjustable back brace is put on by positioning the grid 23 against the user's back then pulling the first end 21 and second end 22 around the user's torso until the Velcro® sections 25, 26 can be secured. In other embodiments, the ends of the brace could be connected by hooks, snaps, buttons, or other means of attachment instead of Velcro®.

The brace 20 can be customized while on the user. The user can reach around his or her body to adjust the desired pins 24. The user can also have a person assist with adjusting the desired pins 24.

The user can alternatively adjust the brace 20 while it is not being worn.

The invention has been described with regard to a back brace. Those skilled in the art understand that the same grid and pin system can be used with braces for other parts of the body, including but no limited to, necks and calves.

I claim:

1. A adjustable brace comprising:
an exterior side, an interior side, a pin grid, and a plurality of adjustment pins;
wherein the pin grid comprises a plurality of threaded pin apertures;
wherein said plurality of adjustment pins have threaded shafts, slotted ends, and domed ends;
wherein the threaded shafts of the adjustment pins form a thread fit with the treaded pin apertures;
wherein the slotted ends of the adjustment pins are proximate the exterior side of the brace and the domed ends of the adjustment pins are proximate the interior side of the brace;
wherein rotating the adjustment pins cause the adjustment pins to incrementally move forward or backward within the pin aperture.

2. The adjustable brace of claim 1 further comprising a first Velcro® section on the exterior side and a second Velcro® section on the interior side wherein said brace is held in place via a Velcro® fit between the two Velcro® sections.

3. A method for applying pressure to body muscles comprising:
using an adjustable back brace comprising an exterior side, an interior side, a pin grid, and a plurality of adjustment pins;
wherein the pin grid comprises a plurality of threaded pin apertures;
wherein said plurality of adjustment pins have threaded shafts, slotted ends, and domed ends;
wherein the threaded shafts of the adjustment pins form a thread fit with the treaded pin apertures;
wherein the slotted ends of the adjustment pins are proximate the exterior side of the brace and the domed ends of the adjustment pins are proximate the interior side of the brace;
wherein rotating the adjustment pins cause the adjustment pins to incrementally move forward or backward within the pin aperture so that the domed end of the pins is pressed into the user's body;
wherein the pin grid is aligned with the area of the body at which pressure is desired;
wherein pins are rotated toward the interior of the brace at positions where pressure is desired;
wherein pins are rotated toward the exterior of the brace at position where pressure is less desired.
